(19) United States
(12) Patent Application Publication
(10) Pub. No.: US 2023/0410953 A1
(43) Pub. Date: Dec. 21, 2023

(54) METHOD FOR CONSTRUCTING AND OPTIMIZING MOLECULAR STRUCTURE MODEL OF LIGNITE

(71) Applicant: Binzhou University, Binzhou (CN)

(72) Inventors: Qingtao Zhang, Binzhou (CN); Xueyang Xing, Binzhou (CN); Yingying Hu, Binzhou (CN); Gang Zhou, Binzhou (CN); Bo Li, Binzhou (CN); Jianjun Shen, Binzhou (CN); Mingming Fu, Binzhou (CN); Xiaofei Li, Binzhou (CN); Lanjuan Xu, Binzhou (CN); Shuai Du, Binzhou (CN); Xinlei Jia, Binzhou (CN); Zhenjiang Yin, Binzhou (CN); Qianqian Zhang, Binzhou (CN); Jie Zang, Binzhou (CN); Qing Cao, Binzhou (CN); Hui Ma, Binzhou (CN); Lei Zhao, Binzhou (CN)

(21) Appl. No.: 18/460,697

(22) Filed: Sep. 5, 2023

Related U.S. Application Data
(63) Continuation of application No. PCT/CN2021/121703, filed on Sep. 29, 2021.

(30) Foreign Application Priority Data
Sep. 23, 2021 (CN) 2021111119809

(51) Int. Cl. G16C 20/70; G16C 20/20; G01N 24/08; G01N 21/35; G06F 30/20

(52) U.S. Cl. CPC G16C 20/70; G16C 20/20; G06F 30/20; G01N 21/35; G01N 24/087

(57) ABSTRACT

The present invention discloses a method for constructing and optimizing a molecular structure model of lignite, comprising: collecting and processing a lignite sample, analyzing a lignite experimental sample, calculating the number of carbon atoms in lignite and the number of carbon atoms in other parameters, obtaining the sizes of aromatic clusters, and determining the composition features and number of aromatic structural units in lignite; calculating the total number of carbon atoms and the number of aliphatic carbon atoms in lignite; obtaining the categories and number of oxygen-containing functional groups in lignite based on the contents of aromatic hydrocarbon, aliphatic hydrocarbon and various oxygen-containing functional groups, and designing the structural forms of nitrogen and sulfur; constructing a molecular structure model of lignite.

### TECHNICAL FIELD

The present invention relates to the technical field of molecular structure characterization of substances, and more particularly to a method for constructing and optimizing a molecular structure model of lignite.

### BACKGROUND

Lignite is a low grade coal which is brownish black and dull between peat and pitch coal, and is also a mineral coal with strong chemical reactivity, easy weathering in air, difficult storage and transportation, and the lowest degree of coalification. As for the research on the molecular structure of lignite, most of the current research results focus on the structure of a specific part, which lack the research on the wetting dynamic features of coal from a microscopic perspective, and cannot essentially explain the wetting mechanism of coal. With the continuous progress and development of computer science, it is not only possible to obtain the structure, dipole moment and ionization energy of coal molecules from computers, but also realize the numerical analysis of the existing mechanical laws by computers, thus to essentially realize the simulation and analysis of microscopic molecular characteristics and behaviors that cannot be completed by experiments.

### SUMMARY

The purpose of the present invention is to provide a method for constructing and optimizing a molecular structure model of lignite, so as to solve the problem in the above prior art, make the constructed molecular structure model of lignite closer to a real structure of coal, and avoid the problem that the research conclusions are similar due to the similarity in structures.

To achieve the above purpose, the present invention provides the following solution: the present invention provides a method for constructing and optimizing a molecular structure model of lignite, comprising:
Collecting and processing a lignite sample to obtain a lignite experimental sample;
Analyzing the lignite experimental sample to obtain the elemental analysis results, the contents of aromatic hydrocarbon, aliphatic hydrocarbon and various oxygen-containing functional groups, and the carbon structural parameters of the lignite sample;
Calculating the number of carbon atoms in lignite based on the elemental analysis results, the contents of aromatic hydrocarbon, aliphatic hydrocarbon and various oxygen-containing functional groups, and the carbon structural parameters, obtaining the sizes of aromatic clusters, and determining the composition features and number of aromatic structural units in lignite;
Calculating the total number of carbon atoms and the number of aliphatic carbon atoms in lignite based on the composition features and number of aromatic structural units;
Obtaining the categories and number of oxygen-containing functional groups in lignite based on the contents of aromatic hydrocarbon, aliphatic hydrocarbon and various oxygen-containing functional groups,
Designing the structural forms of nitrogen and sulfur;
Constructing a molecular structure model of lignite based on the composition features and number of aromatic structural units in lignite, the total number of carbon atoms and the number of aliphatic carbon atoms in lignite, the categories and number of oxygen-containing functional groups in lignite, and the structural forms of nitrogen and sulfur.

Optionally, the step of analyzing the lignite experimental sample comprises:
Conducting infrared spectroscopic analysis to the lignite experimental sample to obtain the contents of aromatic hydrocarbon, aliphatic hydrocarbon and various oxygen-containing functional groups;
Testing the lignite experimental sample by a nuclear magnetic resonance spectrometer through Cross Polarization Magic Angle Spinning (CPMAS) and Total Suppression of Sidebands (TOSS) to obtain the structural parameters.

Optionally, the structural parameters include aromatic carbon, carbonyl carbon with a chemical shift of >165 ppm, aromatic ring carbon, unprotonated carbon, protonated carbon, phenol or aromatic ether carbon, alkylated aromatic carbon, bridging aromatic carbon, aliphatic carbon, aliphatic methyl, aromatic methyl, quaternary carbon, methylene, and aliphatic carbon bonded to oxygen.

Optionally, the sizes of aromatic clusters are obtained by a relation curve of mole fraction x_{b} and number of C atoms per aromatic cluster.

Optionally, in the process of determining the composition features and number of aromatic structural units in lignite, different aromatic structural units are adjusted, optimized and combined, and the ratio of bridging carbon to peripheral carbon after combination is kept consistent with the value in raw coal.

Optionally, the number of carbon atoms in lignite and the number of carbon atoms in other parameters include number of aromatic carbon atoms, number of peripheral carbon atoms, number of bridging carbon atoms, number of alkylated aromatic carbon atoms, number of aliphatic carbon atoms, number of ═CH and ═CH₂ carbon atoms on aliphatic chain, number of unprotonated carbon and —CH₃ carbon atoms on aliphatic chain, and total number of carbon atoms in aromatic clusters.

Optionally, the structural forms of nitrogen include pyrrole and pyridine, and the structural forms of sulfur include mercaptan.

Optionally, the step of constructing a molecular structure model of lignite comprises:
Constructing an initial molecular structure model of lignite based on the composition features and number of aromatic structural units in lignite, the total number of carbon atoms and the number of aliphatic carbon atoms in lignite, the categories and number of oxygen-containing functional groups in lignite, and the structural forms of nitrogen and sulfur;
Conducting ¹³C-NMR prediction and calculation to the initial molecular structure model of lignite, and adjusting the spectral information of a molecular structure model of lignite on the premise of keeping the aromatic units and aromaticity unchanged to construct the molecular structure model of lignite.

The present invention discloses the following technical effects:

Based on the results of infrared spectroscopy experiment, elemental analysis experiment and nuclear magnetic resonance experiment, and in combination with a condensation mode curve, the method for constructing and optimizing a molecular structure model of lignite provided by the present invention specially gives the number and composition of carbon atoms, aromatic unit structures, aliphatic structures, oxygen-containing functional groups and heteroatoms in the molecular structure of lignite, and reconstructs and optimizes the molecular structure of lignite to obtain a molecular structure model of lignite closer to a real structure. The method can elucidate the wetting mechanism of coal dust essentially, provide a theoretical support for the research and development of a wet dust suppression agent, and provide a guarantee for the simulation of post-molecular dynamics behaviors at the same time. The construction of a molecular structure model of coal has a milestone value for the promotion and progress of coal mine dust control technology.

### DESCRIPTION OF DRAWINGS

To more clearly describe the technical solutions in the embodiments of the present invention or in prior art, the drawings required to be used in the embodiments will be simply presented below. Apparently, the drawings in the following description are merely some embodiments of the present invention, and for those skilled in the art, other drawings can also be obtained according to these drawings without contributing creative labor.

FIG. 1 is a flow chart of a method for constructing and optimizing a molecular structure model of lignite in an embodiment of the present invention;

FIG. 2 is a relation curve of mole fraction x_{b} and number of C atoms per aromatic cluster in an embodiment of the present invention;

FIG. 3 shows a planar structural diagram and a ball-and-stick structural diagram of lignite molecules in an embodiment of the present invention, wherein (a) is a planar structural diagram, and (b) is a ball-and-stick structural diagram;

FIG. 4 is a comparison diagram of a ¹³C-NMR experiment spectrum and a prediction spectrum of lignite in an embodiment of the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

To make the above-mentioned purpose, features and advantages of the present invention more clear and understandable, the present invention will be further described below in detail in combination with the drawings and specific embodiments.

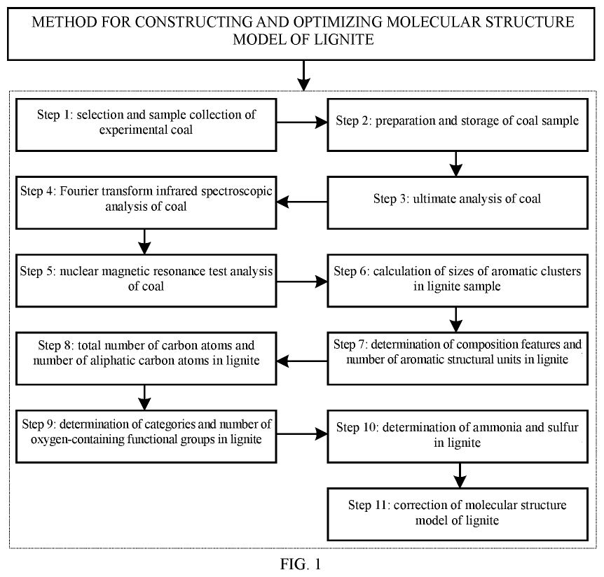

The present invention provides a method for constructing and optimizing a molecular structure model of lignite, as shown in FIG. 1, comprising the following steps:

Step 1: selecting experimental coal and collecting a sample.

In the embodiment, lignite from a coal mine in Shandong province is selected as the research object, and a sample is collected strictly in accordance with the national standard of Method for Manual Sampling Commercial Coal (GB475-2008), packaged, stored and sent to a laboratory. In order to reduce the experimental error caused by coal oxidation, the coal sample is sealed and kept away from light immediately after being taken out, and experiments are carried out within 48 hours after the coal sample is prepared.

Step 2: preparing and storing a lignite experimental sample.

A relatively complete block of coal is selected from the collected coal sample, unedged, and put into a ball mill to prepare coal powders with different particle sizes used in the experiments; in order to improve the crushing efficiency and prevent the coal sample from oxidation due to overheating, the time of each grinding shall not exceed 2 minutes; a coal powder with an appropriate particle size is selected by a test sieve to obtain the lignite experimental sample; the lignite experimental sample is put into a vinyl plastic bottle with nitrogen protection, stored at a low temperature and kept away from light.

Step 3: conducting ultimate analysis to the lignite experimental sample.

Ultimate analysis is conducted to the lignite experimental sample in accordance with the national standard of Ultimate Analysis of Coal (GB/T31391-2015), and the results of the ultimate analysis are shown in Table 1.

TABLE 1

| | | Elemental composition (w %) | | | | | Atomic ratio |
|---|---|---|---|---|---|---|---|
| No. | Coal | C | O | H | N | S | C/H |
| 1# | Lignite | 73.73 | 16.02 | 6.81 | 2.36 | 1.08 | 0.9 |

Step 4: conducting Fourier transform infrared spectroscopic analysis to the lignite experimental sample.

Infrared spectroscopic analysis is conducted to the lignite sample by a NicoletiS20 Fourier transform infrared spectrometer to obtain the contents of aromatic hydrocarbon, aliphatic hydrocarbon and various oxygen-containing functional groups in the coal sample, as shown in Table 2.

TABLE 2

| | | Oxygen-containing functional groups | | | | | | |
|---|---|---|---|---|---|---|---|---|
| | | | | | | Ether-oxygen | | |
| | | Hydroxyl | Carboxyl | Carbonyl | Methoxyl | bond | Aromatic | Aliphatic |
| No. | Coal | (—OH) | (—COOH) | (C═O) | (—OCH₃) | (—O—) | hydrocarbon | hydrocarbon |
| 1# | Lignite | 0.41 | 0.0683 | 0.027 | 0.04 | 0.017 | 0.166 | 0.224 |

Step 5: conducting nuclear magnetic resonance test analysis to the lignite experimental sample.

The lignite sample is tested by an Avance III 400 MHz nuclear magnetic resonance spectrometer from Bruker through Cross Polarization Magic Angle Spinning (CPMAS) and Total Suppression of Sidebands (TOSS) to obtain the structural parameters of the lignite experimental sample, as shown in Table 3.

TABLE 3

| Coal sample | ƒₐ | ƒₐ^{C} | ƒₐ^{'} | ƒₐ^{H} | ƒₐ^{N} | ƒₐ^{P} | ƒₐ^{S} | ƒₐ^{B} | ƒₐₗ | ƒₐₗ^{*} | ƒₐₗ^{H} | ƒₐₗ^{O} |
|---|---|---|---|---|---|---|---|---|---|---|---|---|
| Lignite | 0.61 | 0.03 | 0.58 | 0.42 | 0.17 | 0.06 | 0 | 0.11 | 0.39 | 0.04 | 0.28 | 0.07 |

Note: fₐ-aromatic carbon; fₐ^{C}-carbonyl carbon with a chemical shift of >165 ppm; fₐ′-aromatic ring carbon; fₐ^{N}-unprotonated carbon; fₐ^{H}-protonated carbon; fₐ^{P}-phenol or aromatic ether carbon; fₐ^{S}-alkylated aromatic carbon; fₐ^{B}-bridging aromatic carbon; fₐₗ-aliphatic carbon; fₐₗ*-aliphatic methyl and aromatic methyl; fₐₗ^{H}-quaternary carbon and methylene; fₐₗ^{O}-aliphatic carbon bonded to oxygen.

Step 6: calculating sizes of aromatic clusters in lignite.

Different from other macromolecular organics, the chemical composition and molecular structure morphologies of coal are diversified and complex. Coal has no uniform physical or chemical structure, and the molecular structure and composition morphologies of coal with different metamorphic grades are also obviously different. Therefore, the aromaticity of coal is measured by a solid state NMR experiment method, the structural features of coal are quantitatively characterized by 12 kinds of carbon structures obtained, and the structural parameters of coal measured in the experiments are shown in Table 3.

In order to analyze the structure of coal, the mole fraction x_{b} of bridging aromatic carbon is used in the embodiment. This parameter is a very important index for calculating the sizes of aromatic clusters, and a calculation formula is shown as formula (1).

x_{b}=fₐ^{B}/fₐ′ (1)

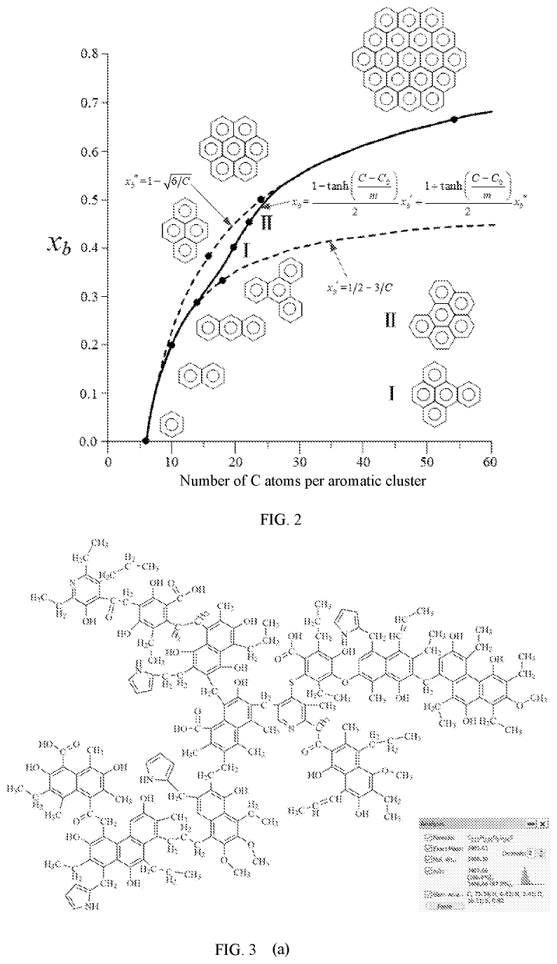

In the embodiment, the relation curve of mole fraction x_{b} and number of C atoms per aromatic cluster (also known as: condensation mode curve) is used to calculate the number of carbon atoms in the lignite experimental sample and the number of carbon atoms in other parameters and obtain the sizes of aromatic clusters. The relation curve of mole fraction x_{b} and number of C atoms per aromatic cluster is shown in FIG. 2, wherein the lower dashed line represents a main chain model, that is to say, when the number of C atoms per cluster is less than 14, the main chain model predominates and is denoted by x_{b}′, and x_{b}′ is shown in formula (2); the upper dashed line represents a ring chain model, that is to say, when the number of C atoms per cluster is greater than 24, the ring chain model predominates and is denoted by x_{b}″, and x_{b}″ is shown in formula (4); when the number of C atoms per cluster is 14-24, a joint model of the two models is used for characterization, which is shown by the solid line in FIG. 2 and denoted by x_{b}, and x_{b} is shown in formula (3);

$${x}_{b}^{′} = 1 / 2 - 3 / C when C \leq 14 \qquad ( 2 )$$

$${x}_{b} = \frac{1 - tanh ( \frac{C - {C}_{0}}{m} )}{2} {x}_{b}^{′} + \frac{1 + tanh ( \frac{C - {C}_{0}}{m} )}{2} {x}_{b}^{″} when \\ 14 \leq C \leq 24 \qquad ( 3 )$$

$${x}_{b}^{″} = 1 - \sqrt{6 / C} when C \geq 14 \qquad ( 4 )$$

Wherein C is the number of carbon atoms, Cₒ is the initial number of carbon atoms, and m is the molar mass.

The number of carbon atoms in the lignite experimental sample and the number of carbon atoms in other parameters are calculated by x_{b} and the relation curve of mole fraction x_{b} and number of C atoms per aromatic cluster in combination with the carbon structural parameters of nuclear magnetic resonance, as shown in Table 4.

TABLE 4

| | Sizes of aromatic clusters | | | | | | | | | | | |
|---|---|---|---|---|---|---|---|---|---|---|---|---|
| Coal sample | Cₐ | C_{P} | C_{B} | C_{S} | Cₐₗ | Cₙ | Cₘ | Rₐ | x_{b} | X_{BP} | C_{T} | Mw |
| Lignite | 9 | 7.32 | 1.68 | 0.92 | 5.64 | 6.41 | 0.61 | 1.84 | 0.186 | 0.229 | 15.25 | 254.59 |

Wherein Cₐ is the number of aromatic carbon atoms, which is mainly calculated according to fₐ′; C_{P} is the number of peripheral carbon atoms, which is mainly calculated according to fₐ^{H}, fₐ^{P} and fₐ^{S}; C_{B} is the number of bridging carbon atoms, which is mainly calculated according to fₐ^{B}; C_{S} is the number of alkylated aromatic carbon atoms, which is mainly calculated according to fₐ^{P} and fₐ^{S}; Cₐₗ is the number of aliphatic carbon atoms, which is mainly calculated according to fₐₗ; Cₙ is the number of ═CH and ═CH₂ carbon atoms on aliphatic chain, which is mainly calculated according to fₐₗ^{H}; Cₘ is the number of unprotonated carbon and —CH₃ carbon atoms on aliphatic chain, which is mainly calculated according to fₐₗ*; C_{T} is the total number of carbon atoms in aromatic clusters, which is calculated by Cₐ/fₐ′; Rₐ is the number of aromatic rings, which is calculated by Rₐ=½ (Cₐ−C_{P})+1, and molecular weight Mw of a single aromatic cluster can be calculated in combination with the elemental composition. In order to better study the construction of the molecular structure model of lignite according to the above steps, the ratio of aromatic bridging carbon to peripheral carbon X_{BP} was introduced, as shown in formula (5):

X_{BP}=fₐ^{B}/(fₐ^{H}+fₐ^{P}+fₐ^{S}) (5)

Step 7: determining the composition features and number of aromatic structural units in lignite.

Among the basic structural units of coal molecules, the irregular parts of benzene, naphthalene and phenanthrene are the basic structural units of lignite with a low metamorphic grade, wherein the aromatic size X_{BP} of naphthalene is 0.25, the aromatic size X_{BP} of anthracene and phenanthrene is 0.4, the aromatic size X_{BP} of pyrene is 0.6, the aromatic size X_{BP} of quaternary aromatic ring is 0.5, and the aromatic size X_{BP} of quintuple aromatic ring is 0.57. In combination with the values for bridging carbon, peripheral carbon and X_{BP} in Table 4, different aromatic structural units are adjusted, optimized and combined to make the ratio of bridging carbon to peripheral carbon after combination kept consistent with the value in raw coal; based on this principle, the composition features of aromatic units in the coal to be studied are determined; meanwhile, continuous adjustment is required; and finally, the composition features of aromatic structural units in a molecular model of lignite are obtained, as shown in Table 5.

TABLE 5

| Structures types of aromatic units | 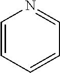 | 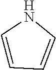 | 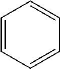 | 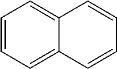 | 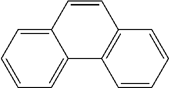 |
|---|---|---|---|---|---|
| Lignite | 2 | 4 | 2 | 6 | 2 |

Step 8: calculating the total number of carbon atoms and the number of aliphatic carbon atoms in lignite.

The total number of carbon atoms and the number of aliphatic carbon atoms in the lignite experimental sample are calculated according to the total number of aromatic carbon atoms in aromatic structural units calculated in step 7 in combination with the structural parameters of the lignite experimental sample.

Aliphatic structures are the main crosslink bonds in coal and has an important influence on many characteristics of coal; the aliphatic structures in coal mainly exists in the forms of alkyl side chains (groups such as methyl, methylene and ethyl) alicyclic hydrocarbons (or hydrogenated aromatic hydrocarbons) and bridge bonds connecting aromatic clusters. With the increase of the metamorphic grade of the coal, the aromatization grade of the coal is gradually increased, and the alkyl side chains are decreased; the value of quaternary carbon and methylene (fₐₗ^{H}) is greater than the value of methyl (fₐₗ*), indicating that quaternary carbon and methylene are widely distributed in aliphatic carbon atoms in the molecular structure; when the carbon content is 80.4%, the average number of carbon atoms in the alkyl side chains is 2.2; and when the carbon content is 84.3%, the average number of carbon atoms in the alkyl side chains is 1.8.

According to that the total number of aromatic carbon atoms in aromatic structural units listed in Table 5 is 126, and in combination with the ratio of aromatic carbon to aliphatic carbon in Table 3, the number of aliphatic carbon atoms is determined to be about 87, so that the total number of carbon atoms in lignite is calculated as 213.

Step 9: determining the categories and number of oxygen-containing functional groups in lignite.

The categories and number of oxygen-containing functional groups in lignite are determined in combination with the contents of aromatic hydrocarbon, aliphatic hydrocarbon and various oxygen-containing functional groups in lignite obtained by the infrared spectroscopy experiment and analysis in step 4.

The oxygen-containing functional groups of coal are mainly in five basic forms, including hydroxyl (—OH), carboxyl (—COOH), carbonyl (C═O), methoxyl (—OCH₃) and ether-oxygen bond (—O—); according to the ratio of carbon to oxygen in the results of elemental analysis and the total number of carbon atoms in lignite (213), the number of oxygen atoms is determined to be about 36 by 213×16.02÷16÷73.73÷12; then, according to the different contents of the five forms of oxygen-containing functional groups analyzed in combination with the infrared spectroscopy experiment, continuous adjustment, optimization and combination are conducted to obtain the categories and number of oxygen-containing functional groups in lignite, as shown in Table 6.

TABLE 6

| Different | | | | | |
|---|---|---|---|---|---|
| categories of | | | | | |
| oxygen- | | | | | Ether- |
| containing | | | | | oxygen |
| functional | Hydroxyl | Carboxyl | Carbonyl | Methoxyl | bond |
| groups | (—OH) | (—COOH) | (C═O) | (—OCH₃) | (—O—) |
| Lignite | 22 | 4 | 3 | 3 | 1 |

Step 10: determining ammonia and sulfur in lignite.

In the structure of the coal, nitrogen mainly exists in the forms of pyrrole and pyridine; in addition, a tiny amount of quinoline, indole, amidocyanogen and nitrile groups may also be contained; as the amount of such groups is tiny, the nitrogen in the embodiment exists only in the forms of pyrrole and pyridine, and the other groups are ignored; according to the ratio of C to N in the results of elemental analysis, it can be determined that the number of nitrogen atoms is about 6, then the numbers of pyrrole and pyridine atoms are adjusted, the number of pyrrole atoms is finally determined to be 4, and the number of pyridine atoms is finally determined to be 2. It can be seen from the elemental analysis of the coal that the content of sulfur is relatively low in the molecular structure of the coal, and organic sulfur exists in the form of thioether in the lignite, which is constructed in the form of mercaptan when the molecular structure model is constructed. According to the ratio of C to S in the results of elemental analysis, it can be determined that the number of sulfur atoms is about 1.

Step 11: constructing and correcting a molecular structure model of lignite.

In the embodiment, the numbers of various atoms in lignite molecules and the molecular formula are finally determined, as shown in Table 7:

TABLE 7

| Number of different | Aromatic | Aliphatic | Total | | | | | Molecular |
|---|---|---|---|---|---|---|---|---|
| categories of atoms | carbon | carbon | carbon | Hydrogen | Nitrogen | Oxygen | Sulfur | formula |
| Lignite | 126 | 87 | 213 | 236 | 6 | 36 | 1 | C₂₁₃H₂₃₆N₆O₃₆S |

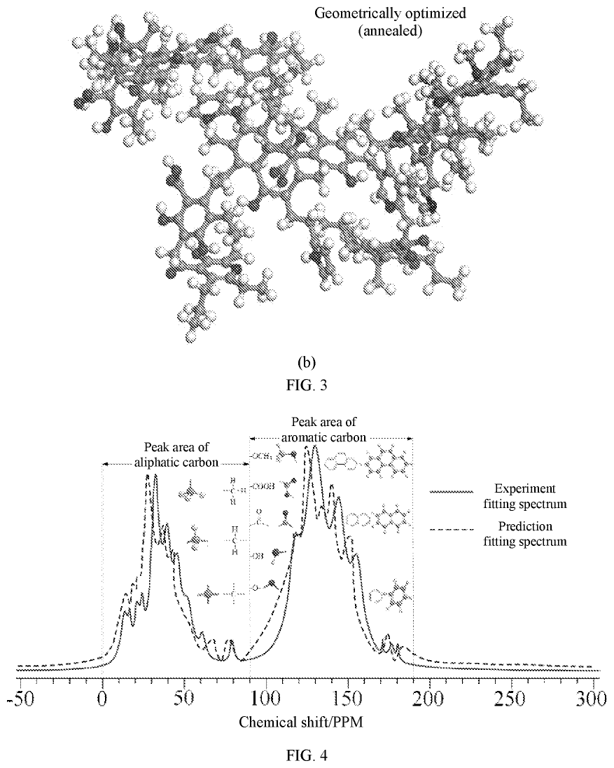

In the embodiment, low-molecular compounds in coal are not considered, the obtained information are tested by basic experiments such as infrared spectroscopy experiment and nuclear magnetic resonance experiment, the numbers of various atoms in lignite molecules and the molecular formula are finally determined, and an initial molecular structure model of lignite is constructed based on the characteristics and ideas of the Wiser chemical structure model; in order to make the structure of the constructed model closer to the real structure of coal, ¹³C-NMR prediction and calculation are conducted to the initial structure model in a Chem Draw software to obtain initial spectral information, and continuous adjustment is conducted to make the spectral information closer to the spectral information of the original experiments; in the process of structural adjustment, the aromatic units and aromaticity are kept unchanged to ensure the accuracy of the skeleton carbon atoms; after continuous adjustment, the spectral information finally obtained is compared with the spectral information obtained in the original experiments by a Spectrus processor software in ACD/Labs (Advanced Chemistry Development); the coincidence degree is good, indicating that the constructed molecular structure of coal is a good reflection of the real structure thereof. The constructed molecular structure of lignite (including a planar molecular structure (a) and a three-dimensional ball-and-stick model (b)) is shown in FIG. 3; comparison of a ¹³C-NMR experiment spectrum and a prediction spectrum of lignite molecules is shown in FIG. 4.

After the construction and correction of the molecular structure model of coal and the comparison of the ¹³C-NMR experiment spectrum and the prediction spectrum, the experiment spectrum is basically consistent with the constructed prediction spectrum, indicating that the constructed molecular structure basically restores the real internal structure of coal, so that the research has not only a certain pertinence, but also an on-site guidance significance.

The above embodiments only describe preferred embodiments of the present invention, and are not intended to limit the scope of the present invention. On the premise of not departing from the design spirit of the present invention, various modifications and improvements made to the technical solution of the present invention by those ordinary skilled in the art shall fall into the protection scope determined by claims of the present invention.

1. A method for constructing and optimizing a molecular structure model of lignite, comprising:
collecting and processing a lignite sample to obtain a lignite experimental sample;
analyzing the lignite experimental sample to obtain the elemental analysis results, the contents of aromatic hydrocarbon, aliphatic hydrocarbon and various oxygen-containing functional groups, and the carbon structural parameters of the lignite sample;
calculating the number of carbon atoms in lignite based on the elemental analysis results, the contents of aromatic hydrocarbon, aliphatic hydrocarbon and various oxygen-containing functional groups, and the carbon structural parameters, obtaining the sizes of aromatic clusters, and determining the composition features and number of aromatic structural units in lignite;
calculating the total number of carbon atoms and the number of aliphatic carbon atoms in lignite based on the composition features and number of aromatic structural units;
obtaining the categories and number of oxygen-containing functional groups in lignite based on the contents of aromatic hydrocarbon, aliphatic hydrocarbon and various oxygen-containing functional groups,
designing the structural forms of nitrogen and sulfur;
constructing a molecular structure model of lignite based on the composition features and number of aromatic structural units in lignite, the total number of carbon atoms and the number of aliphatic carbon atoms in lignite, the categories and number of oxygen-containing functional groups in lignite, and the structural forms of nitrogen and sulfur.

2. The method for constructing and optimizing a molecular structure model of lignite according to claim 1, wherein the step of analyzing the lignite experimental sample comprises:
conducting infrared spectroscopic analysis to the lignite experimental sample to obtain the contents of aromatic hydrocarbon, aliphatic hydrocarbon and various oxygen-containing functional groups;
testing the lignite experimental sample by a nuclear magnetic resonance spectrometer through Cross Polarization Magic Angle Spinning (CPMAS) and Total Suppression of Sidebands (TOSS) to obtain the structural parameters.

3. The method for constructing and optimizing a molecular structure model of lignite according to claim 1, wherein the structural parameters include aromatic carbon, carbonyl carbon with a chemical shift of >165 ppm, aromatic ring carbon, unprotonated carbon, protonated carbon, phenol or aromatic ether carbon, alkylated aromatic carbon, bridging aromatic carbon, aliphatic carbon, aliphatic methyl, aromatic methyl, quaternary carbon, methylene, and aliphatic carbon bonded to oxygen.

4. The method for constructing and optimizing a molecular structure model of lignite according to claim 1, wherein the sizes of aromatic clusters are obtained by a relation curve of mole fraction x_{b} and number of C atoms per aromatic cluster.

5. The method for constructing and optimizing a molecular structure model of lignite according to claim 1, wherein in the process of determining the composition features and number of aromatic structural units in lignite, different aromatic structural units are adjusted, optimized and combined, and the ratio of bridging carbon to peripheral carbon after combination is kept consistent with the value in raw coal.

6. The method for constructing and optimizing a molecular structure model of lignite according to claim 1, wherein the number of carbon atoms in lignite and the number of carbon atoms in other parameters include number of aromatic carbon atoms, number of peripheral carbon atoms, number of bridging carbon atoms, number of alkylated aromatic carbon atoms, number of aliphatic carbon atoms, number of ═CH and ═CH₂ carbon atoms on aliphatic chain, number of unprotonated carbon and —CH₃ carbon atoms on aliphatic chain, and total number of carbon atoms in aromatic clusters.
